(19) United States
(12) Patent Application Publication
(10) Pub. No.: US 2019/0247568 A1
(43) Pub. Date: Aug. 15, 2019

(54) Medical Infusion Pump System

(71) Applicant: Michael George Thatcher, South Jordan, UT (US)

(72) Inventor: Michael George Thatcher, South Jordan, UT (US)

(21) Appl. No.: 15/892,769

(22) Filed: Feb. 9, 2018

(51) Int. Cl. A61M 5/14; A61M 5/172; A61M 5/145; A61K 38/28; A61K 38/22

(52) U.S. Cl. CPC A61M 5/1408; A61M 5/1723; A61M 5/1452; A61K 38/28; A61M 2205/502; A61M 2230/201; A61M 2205/52; A61M 2205/18; A61M 2205/3592; A61K 38/22

(57) ABSTRACT

A medical infusion pump system is disclosed. The medical infusion pump system is a multi-reservoir infusion device for dispensing at least compositions of insulin and an amylin analog for treatment of subjects. Insulin and the amylin analog are stored in separate reservoirs, each reservoir being operatively connected to a motor for providing discharge pressure to the reservoirs. The motors may act independently such that insulin and the amylin analog may be injected at incongruent times. The medical infusion pump may automatically dispense the amylin analog into a subject's blood once the subject's blood-glucose levels rise above a specific threshold, or may dispense the amylin analog based on expected levels of blood glucose.

### CROSS REFERENCE TO RELATED APPLICATIONS

This application claims priority to U.S. Provisional Patent Application No. 62/456,681, filed Feb. 9, 2018, entitled “Medical Infusion Pump System,” which is hereby incorporated by reference in its entirety.

### TECHNICAL FIELD

The present disclosure relates generally to a medical infusion pump system. Specifically, the present disclosure relates to an infusion pump system that provides subjects with amylin and insulin.

### BRIEF DESCRIPTION OF THE DRAWINGS

The written disclosure herein describes illustrative embodiments that are non-limiting and non-exhaustive. Reference is made to certain of such illustrative embodiments that are depicted in the figures described below.

FIG. 1A is an embodiment of a multi-reservoir infusion device with a tubing system for dispensing insulin and an amylin analog (“amylin”);

FIG. 1B is an isometric view of the multi-reservoir infusion device of FIG. 1A with the external casing removed;

FIG. 1C is an exploded view of the multi-reservoir infusion device of FIG. 1B;

FIG. 2A is a wireless communication system, according to one embodiment, comprising a multi-reservoir infusion device that includes a microcontroller, a noninvasive blood-glucose monitoring mechanism, and a personal electronic device;

FIG. 2B is an embodiment of the wireless enabled personal electronic device of FIG. 2A;

FIG. 2C is an embodiment of the noninvasive blood-glucose monitoring mechanism of FIG. 2A;

FIG. 3 is a functional block diagram for a medical infusion pump system, according to one embodiment; and

FIG. 4 is a functional block diagram for a medical infusion pump system, according to another embodiment.

### DETAILED DESCRIPTION

Diabetes mellitus is a disease of major global importance. In healthy individuals, blood glucose concentration is maintained within the normal range, 70-180 mg/dl, by insulin, a hormone produced by the pancreatic beta cells. In individuals with Type 1 Diabetes, the absence of insulin secretion may lead to a high concentration of blood glucose. While hyperglycemia (BG>180 mg/dl) can produce long-term complications (e.g. cardiovascular disease, neuropathy, nephropathy and retinopathy), hypoglycemia (BG<70 mg/dl) is dangerous in the short-term (e.g. seizure, coma and death). Conventional Type 1 Diabetes therapy is based on insulin administrations, performed by the patient himself through multiple daily injections, or continuous subcutaneous insulin infusion, according to a basal-bolus insulin regimen in which insulin boluses are injected when an expected increase in carbohydrates and/or blood glucose levels is to occur. Basal insulin may be administered to maintain a normal glucose level in absence of meal perturbation. The timing of insulin boluses can help lower hyperglycemic peaks.

In healthy individuals, the hormone amylin may be secreted along with insulin in response to food by the pancreatic beta cells. Amylin secretion may suppress glucagon secretion after meals, which regulates blood glucose levels. Some patients with Type 1 Diabetes may develop a deficiency of both insulin and amylin. Injection of an amylin analog, such as Pramlintide or Pramlintide Acetate—also referred to as Symlin, may slow the rate at which food is released from the stomach to the small intestine after a meal. The slower release of food leads to a reduced postprandial rise of blood glucose.

Disclosed herein is a medical infusion pump system for pumping both insulin and an amylin analog into a subject's bloodstream for the purpose of improving and/or stabilizing their blood glucose levels. The disclosed embodiments include a multi-reservoir infusion device for pumping insulin and the amylin analog that includes a noninvasive blood glucose monitoring mechanism.

As used herein, the term “basal” may refer to a minimum required rate or other value for something to function. For example, with respect to insulin therapy the term “basal rate” can refer to a regular (e.g., in accordance with fixed order or procedure, such as regularly scheduled for/at a fixed time), periodic, or continuous delivery of low levels of insulin, such as but not limited to throughout a 24-hour period. The term “basal rate profile,” as used herein, may refer to an insulin delivery schedule that includes one or more blocks of time, wherein each block defines an insulin delivery rate.

Exemplary embodiments disclosed herein relate to the use of a continuous glucose monitor (CGM) that measures a concentration of glucose. In some embodiments, the device can analyze a plurality of intermittent blood samples. The CGM can use any method of glucose measurement, including enzymatic, chemical, physical, electrochemical, optical, optochemical, fluorescence-based, spectrophotometric, spectroscopic (e.g., optical absorption spectroscopy, Raman spectroscopy, etc.), polarimetric, calorimetric, iontophoretic, radiometric, and the like The CGM may provide a data stream to the multi-reservoir infusion device. The data stream is typically a raw data signal that is used to provide a useful value for blood-glucose levels to a user, such as a patient or health care professional (e.g., doctor) that may be using the CGM. In various embodiments, the CGM may include one or more continuous glucose monitoring sensors that may measure, almost continuously (e.g., every 1-5 minutes), interstitial glucose concentration in the subcutaneous tissue.

The medical infusion pump system may include a processor and/or microprocessor that may be configured to coordinate one or more desired functions (e.g., measure, compare, analyze, normalize, etc.). A processor may run a standard operating system and perform standard operating system functions. It is recognized that any standard operating system may be used, such as, for example, Google Android®, Microsoft® Windows®, Apple® MacOS®, Disk Operating System (DOS), UNIX, IRJX, Solaris, SunOS, FreeBSD, Linux®, QNX®, ffiM® OS/2® operating systems, and so forth. The term “processor module,” as used herein, may refer to a computer system, state machine, processor, or the like designed to perform mathematical calculations or logic operations using logic circuitry.

As used herein, the phrases “coupled” or “in communication with” are broad enough to refer to any suitable coupling or other form of interaction between two or more components, including wireless, hardwire, electrical, mechanical, thermal, or the like. Two components may be in communication with each other even though there may be a physical space or intermediary devices between components.

Aspects of certain embodiments described herein may be implemented as software modules or components. As used herein, a software module or component may include any type of computer instruction or computer executable code located within or on a computer-readable storage medium. A software module may, for instance, comprise one or more physical or logical blocks of computer instructions, which may be organized as a routine, program, object, component, data structure, etc., that perform one or more tasks or implement particular abstract data types. A particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media.

In certain embodiments, a particular software module or component may comprise disparate instructions stored in different locations of a memory device, which together implement the described functionality of the module. Indeed, a module or component may comprise a single instruction or many instructions, and may be distributed over several different code segments, among different programs, and across several memory devices. Some embodiments may be practiced in a distributed computing environment where tasks are performed by a remote processing device linked through a communications network. In a distributed computing environment, software modules or components may be located in local and/or remote memory storage devices. In addition, data being tied or rendered together in a database record may be resident in the same memory device, or across several memory devices, and may be linked together in fields of a record in a database across a network.

Any “communications network” or “network” disclosed herein may include a wide variety of network infrastructures. Specifically, a network may incorporate landlines, wireless communication, optical connections, various modulators, demodulators, small form-factor pluggable (SFP) transceivers, routers, hubs, switches, and/or other networking equipment. The network may include communications or networking software, such as software available from Novell, Microsoft, Artisoft, and other vendors, and may operate using TCP/IP, SPX, IPX, SONET, ATT, and other protocols over twisted pair cables, coaxial cables, optical fiber cables, telephone lines, satellites, microwave relays, modulated AC power lines, physical media transfer, wireless radio links, and/or other data transmission “wires.” The network may encompass smaller networks and/or be connectable to other networks through a gateway or similar mechanism.

Some embodiments may be practiced in a distributed computing environment where tasks are performed by a remote processing device linked through a communications network. In a distributed computing environment, software modules may be located in local and/or remote computer-readable storage media. In addition, data being tied or rendered together in a database record may be resident in the same computer-readable storage medium, or across several computer-readable storage media, and may be linked together in fields of a record in a database across a network. According to one embodiment, a database management system (DBMS) allows users to interact with one or more databases and provides access to the data contained in the databases.

Computers may comprise one or more modules. A module may include all or portions of other elements of the system. The modules may run multiple operations concurrently or in parallel by or on one or more processors. Portions of the disclosed modules, components, and/or facilities are embodied as executable instructions embodied in hardware or firmware, or stored on a non-transitory, machine-readable storage medium. The instructions may comprise computer program code that, when executed by a processor and/or computing device, causes a computing system to implement certain processing steps, procedures, and/or operations, as disclosed herein. The modules, components, and/or facilities disclosed herein may be implemented and/or embodied as a driver, a library, an interface, an API, FPGA configuration data, firmware (e.g., stored on an EEPROM), and/or the like. Portions of the modules, components, and/or facilities disclosed herein are embodied as machine components, such as general and/or application-specific devices, including, but not limited to: circuits, integrated circuits, processing components, interface components, hardware controller(s), storage controller(s), programmable hardware, FPGAs, ASICs, and/or the like. Accordingly, the modules disclosed herein may be referred to as controllers, layers, services, engines, facilities, drivers, circuits, and/or the like.

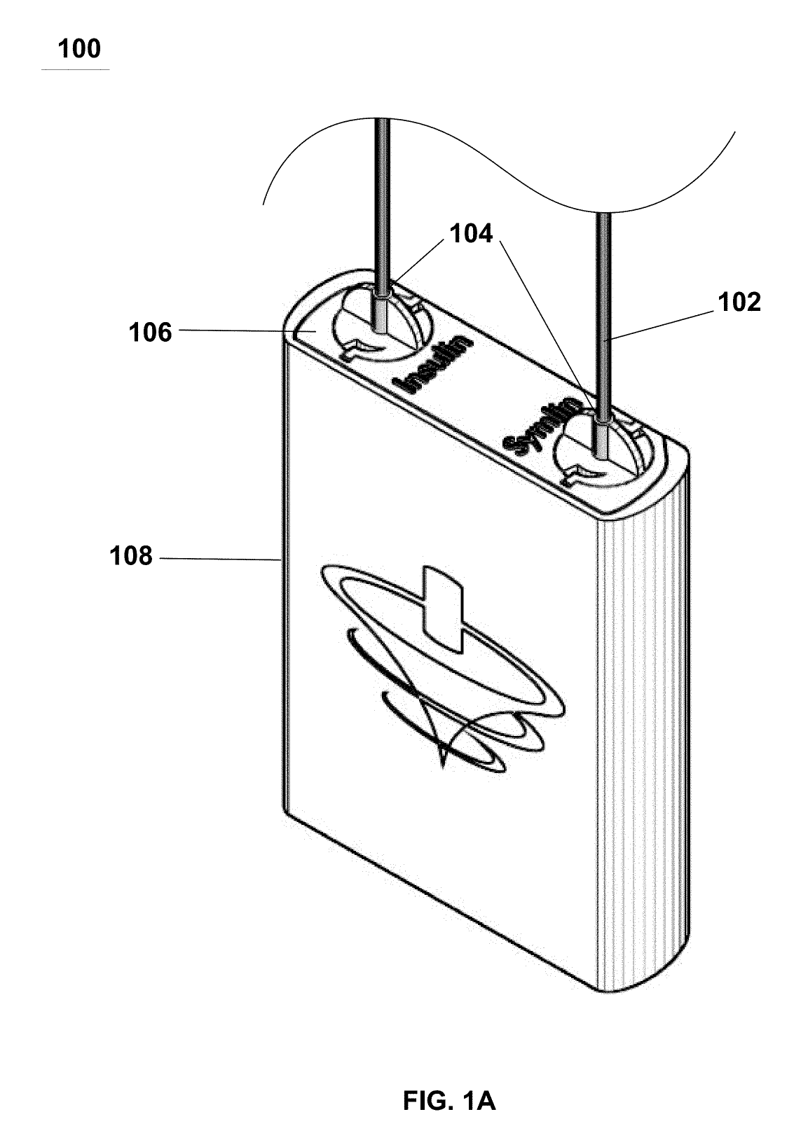

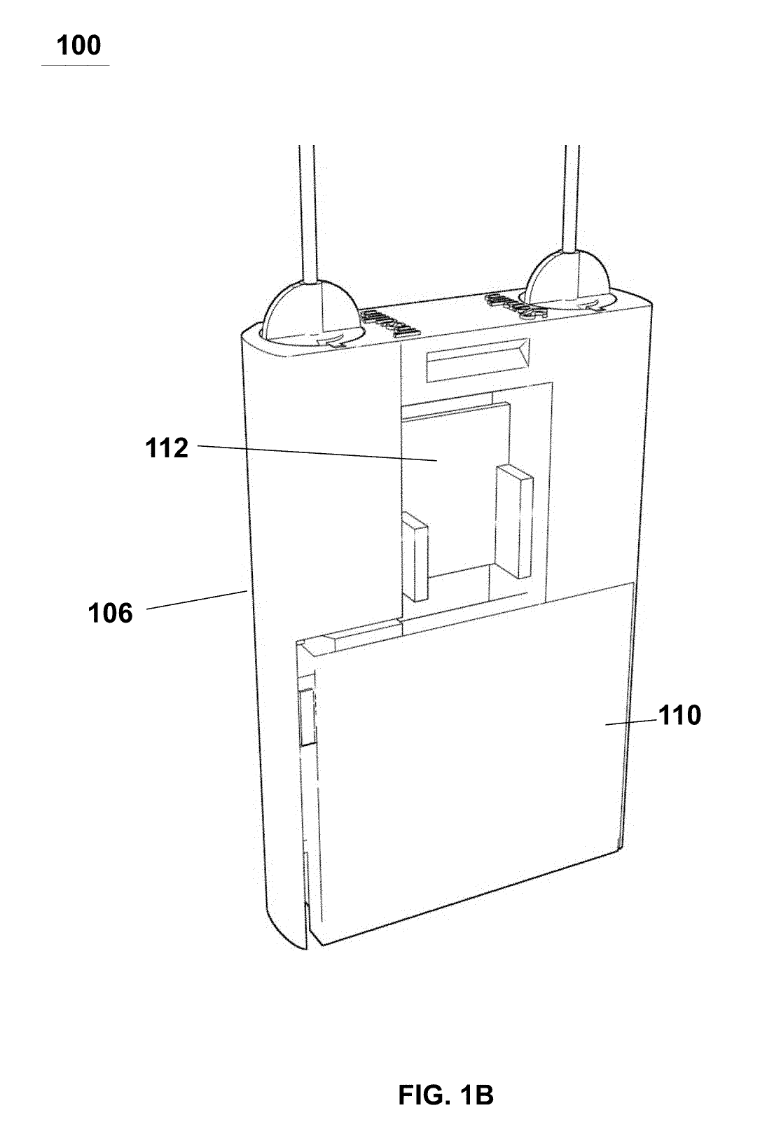

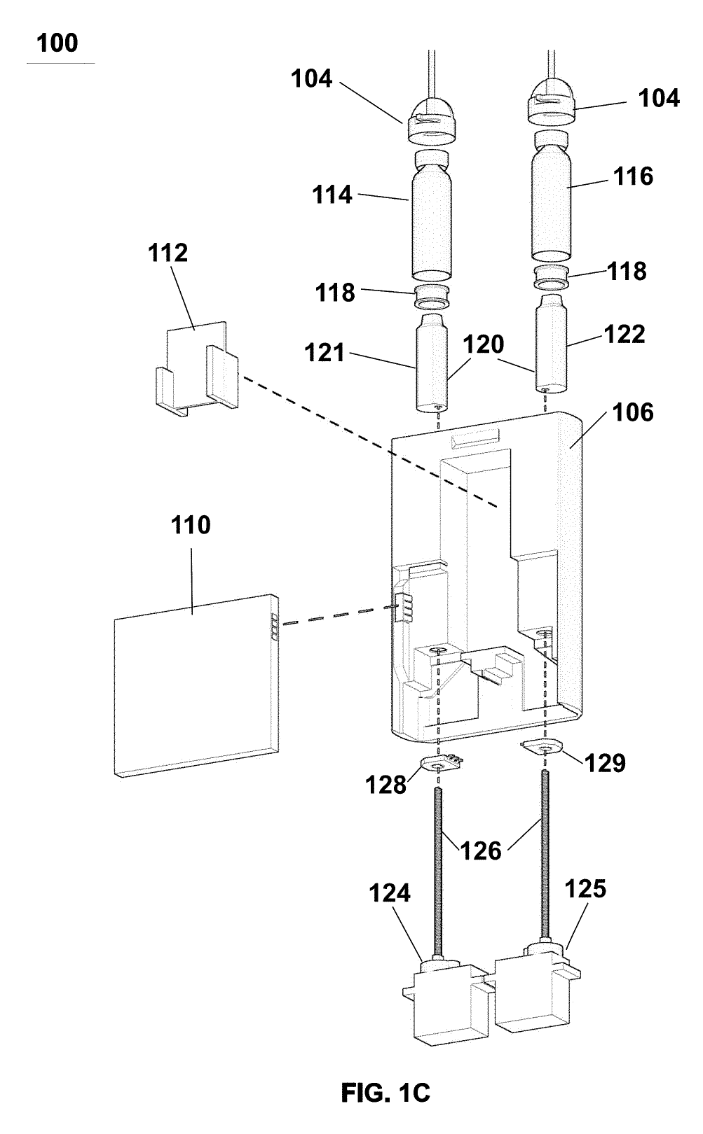

FIG. 1A is an embodiment of a multi-reservoir infusion device 100 with a tubing system 102 for dispensing insulin and an amylin analog (“amylin”). The infusion device 100 may include an infusion set 104 to which the tubing system 102 is coupled. The infusion set 104 may be fastened to a housing 106 for the infusion device 100 that is surrounded by an external casing 108. The infusion device 100 may be part of a medical infusion pump system for injecting insulin and amylin through the tubing system and into a subject's bloodstream.

In one embodiment, the tubing system 102 may include a cannula for subcutaneous insertion and a tubing system for interfacing the multi-reservoir infusion device 100 to the cannula. Specifically, the tubing system may interface with an insulin reservoir and/or amylin reservoir located within the infusion device 100. The tubing system 102 itself, as well as components of the tubing system 102, may be disposable such that a replacement tubing system 102 may be coupled to the infusion device 100 upon failure and/or wear of the tubing system 102.

FIG. 1B is an isometric view of the multi-reservoir infusion device 100 of FIG. 1A with the external casing (see FIG. 1A) removed. The housing 106 may support an internal power source 110 as well as a wireless enabled microcontroller 112. The power source 110, according to various embodiments, may be a lithium polymer battery, lithium ion battery, nickel metal hydride battery, nickel cadmium battery, or the like. The wireless enabled microcontroller 112 may be a small computer on an integrated circuit comprising one or more computer processing units (CPUs), memory, and input/output peripherals.

The microcontroller 112 may include a component for and/or be operatively connected to a computer for calculating how much insulin to automatically inject based on expected carbohydrate intake, blood sugar level, still-active insulin and/or other indicators that determine how much insulin the subject needs, according to various embodiments. In various embodiments, the computer may be part of a network. The microcontroller 112 may also include a memory storage for recording a record of insulin and amylin usage and from which the computer may obtain input data, computations, recordings, and/or other stored information. The microcontroller may comprise or be operatively connected to a computation software module for computing future blood glucose levels of a subject.

The microcontroller 112 may also be operatively connected to and/or comprise an amylin monitor for determining how much of the amylin has been injected and adjusting future insulin dose levels based on an amount of injected amylin. A timer for monitoring an amount of time elapsed after insulin delivery may also be operatively connected to or included in the microcontroller 112, according to various embodiments.

The microcontroller 112 may be operatively connected to a carbohydrate counter, which may store data in a memory. A processor module may comprise a programmed basal rate profile for insulin delivery and be operatively connected to or included in an embodiment of the microcontroller 112. In one embodiment, the microcontroller 112 may be operatively connected to an automated tracker for tracking a subject's previous blood-glucose patterns and preprogramming future insulin and/or amylin quantities based on the patterns.

FIG. 1C is an exploded view of the multi-reservoir infusion device 100 of FIG. 1B. The housing 106 may provide support to various components of the infusion pump system. The infusion device 100 may dispense at least compositions of insulin and an amylin for treatment of subjects. The insulin may be stored in an insulin reservoir 114 that is operatively connected to the infusion set 104. The amylin may be stored in an amylin reservoir 116 and is also operatively connected to the infusion set 104. In one embodiment, the insulin reservoir 114, and/or amylin reservoir 116 are disposable. Cartridge plungers 118 may separate the insulin reservoir 114 and the amylin reservoir 116 from two or more positive displacement pumps 120. The positive displacement pumps 120 may include an insulin piston 121 and an amylin piston 122, according to one embodiment. In another embodiment, the positive displacement pumps 120 may be peristaltic pumps.

The multi-reservoir infusion device 100 may comprise a microcontroller 112 operatively connected to an insulin motor 124 and an amylin motor 125, according to one embodiment. The insulin motor 124 may correspond to the insulin piston 121 and the amylin motor 125 may correspond to the amylin piston 122. The insulin motor 124 may be coupled to a leadscrew 126, an insulin encoder 128, and an insulin piston 121. Similarly, the amylin motor 125 may be coupled to a leadscrew 126, an amylin encoder 129, and an amylin piston 122. A power source 110 may be operatively connected to the insulin motor 124, amylin motor 125, and the microcontroller 112.

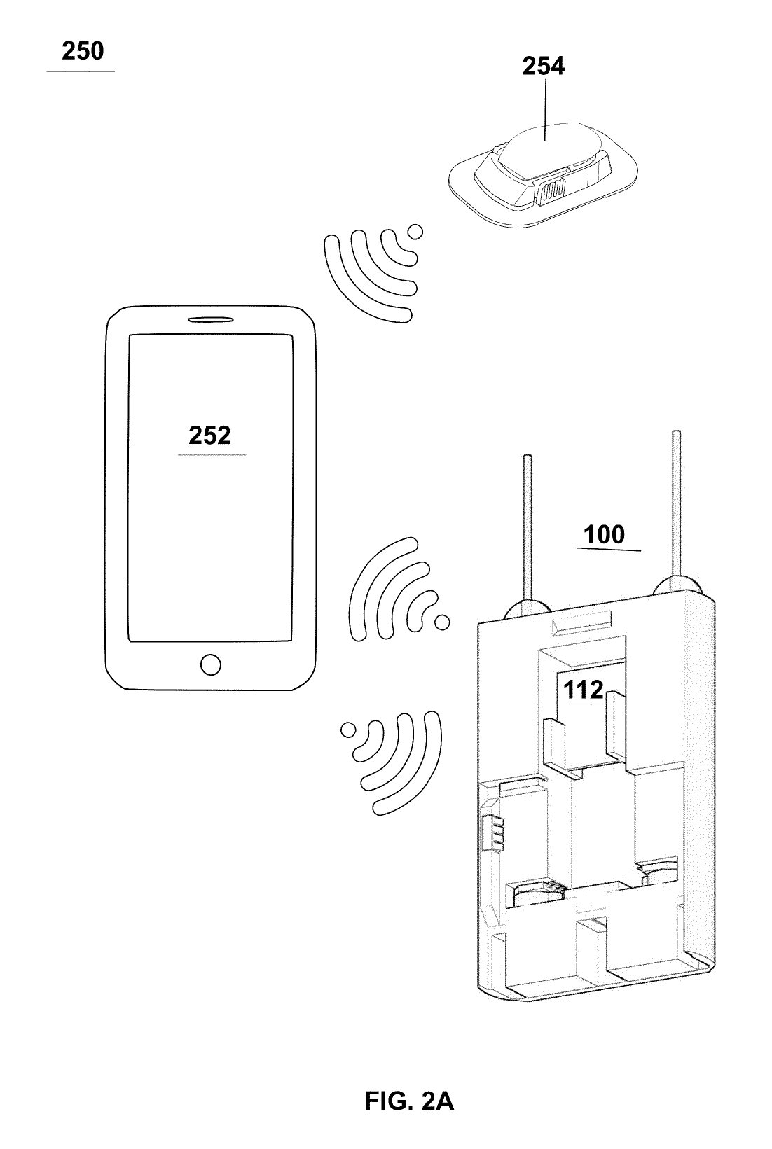

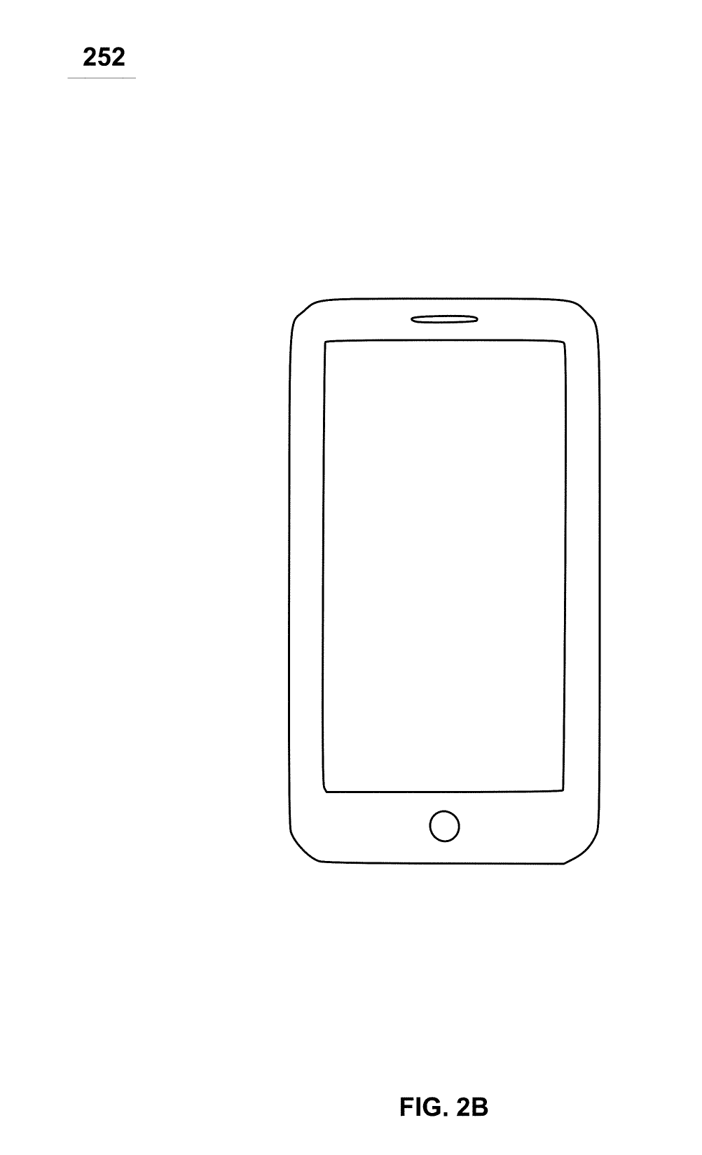

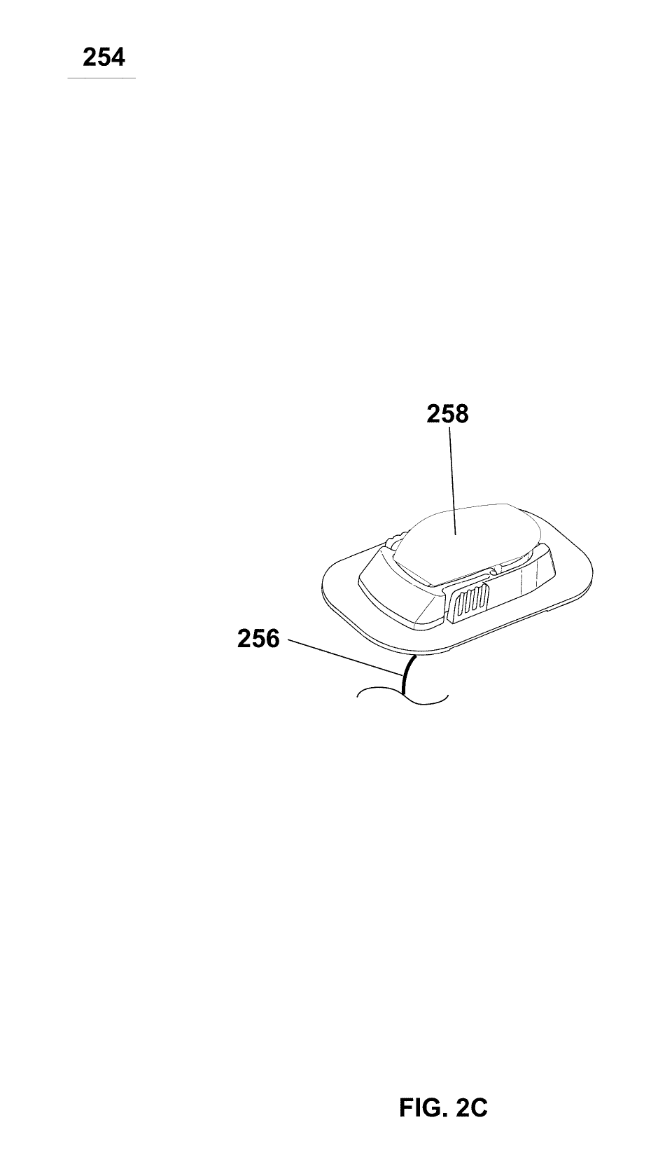

FIG. 2A is a wireless communication system 250, according to one embodiment, comprising a multi-reservoir infusion device 100 that includes a microcontroller 112, a continuous glucose monitor (CGM) 254, and a personal electronic device 252. The wireless communication system 250 may be a wireless personal area network (WPAN) (e.g., Bluetooth), a wireless local area network (WLAN), a mobile ad hoc network (MANET), wireless metropolitan area network, wireless wide area network, global area network, or the like. In one embodiment, the wireless communication system 250 may provide an alert mechanism for alerting a subject of any malfunctions and/or other irregularities in the pump system's performance.

The personal electronic device 252 may be a mobile phone, tablet, laptop computer, or the like, according to various embodiments. In one embodiment, the personal electronic device 252 may include a user interface operatively connected to a processor. The user interface may include a monitor or other display, printer, speech or text synthesizer, graphical user interface, or other hardware with accompanying firmware and/or software. The personal electronic device 252 may comprise one or more input/output interfaces that facilitate user interfacing. The input interface(s) may include a keyboard, mouse, button, touch screen, light pen, tablet, microphone, sensor, or other hardware with accompanying firmware and/or software. The personal electronic device 252 may include one or more software modules and/or processor modules for providing instructions and calculations to the multi-reservoir infusion device 100.

The CGM 254 may continually monitor glucose levels in a subject's blood. In one embodiment, the personal electronic device 252 may comprise a trigger, wherein the trigger automatically causes the multi-reservoir infusion device 100 to inject a bolus of insulin in response to real time continuous glucose data received from the CGM 254. The trigger may also automatically determine the amount of amylin to deliver in real time based on data obtained from the CGM 254. In one embodiment, the multi-reservoir infusion device 100 may automatically dispense amylin into the subject's blood once the subject's blood-glucose levels rise above an expected threshold determined by an amount of insulin previously delivered to the subject. In one embodiment, a subject and/or a medical professional manually set the expected threshold. In another embodiment, the personal electronic device 252 may signal the multi-reservoir infusion device to dispense amylin at periodic time increments independent of any insulin levels.

FIG. 2B is an embodiment of the wireless enabled personal electronic device 252 of FIG. 2A. The personal electronic device 252 may comprise a software application or software module for receiving user inputs and transmitting user outputs. In one embodiment the software application may perform one or more logic functions responsive to inputs received from the noninvasive CGM (see FIG. 2A). The personal electronic device 252 may display quantitative images representing blood glucose levels. In one embodiment, the personal electronic device may provide a visual, auditory, and/or other sensory alert to a subject for when blood glucose levels deviate from an acceptable range.

FIG. 2C is an embodiment of the noninvasive CGM 254 of FIG. 2A. The CGM 254 may provide an output of real-time blood glucose levels, the rate at which blood glucose levels are increasing or decreasing, and monitor trends in blood glucose levels. In various embodiments, the CGM 254 may provide hundreds or thousands of blood glucose readings each day. One embodiment of the CGM 254 may comprise a sensor 256 that measures glucose levels subdermally. The sensor 256 may be disposable, according to one embodiment, and replaced periodically with a new sensor 256. The CGM 254 may also include a transmitter 258 coupled to the sensor 256 to transmit data within a wireless communication system (see FIG. 2A).

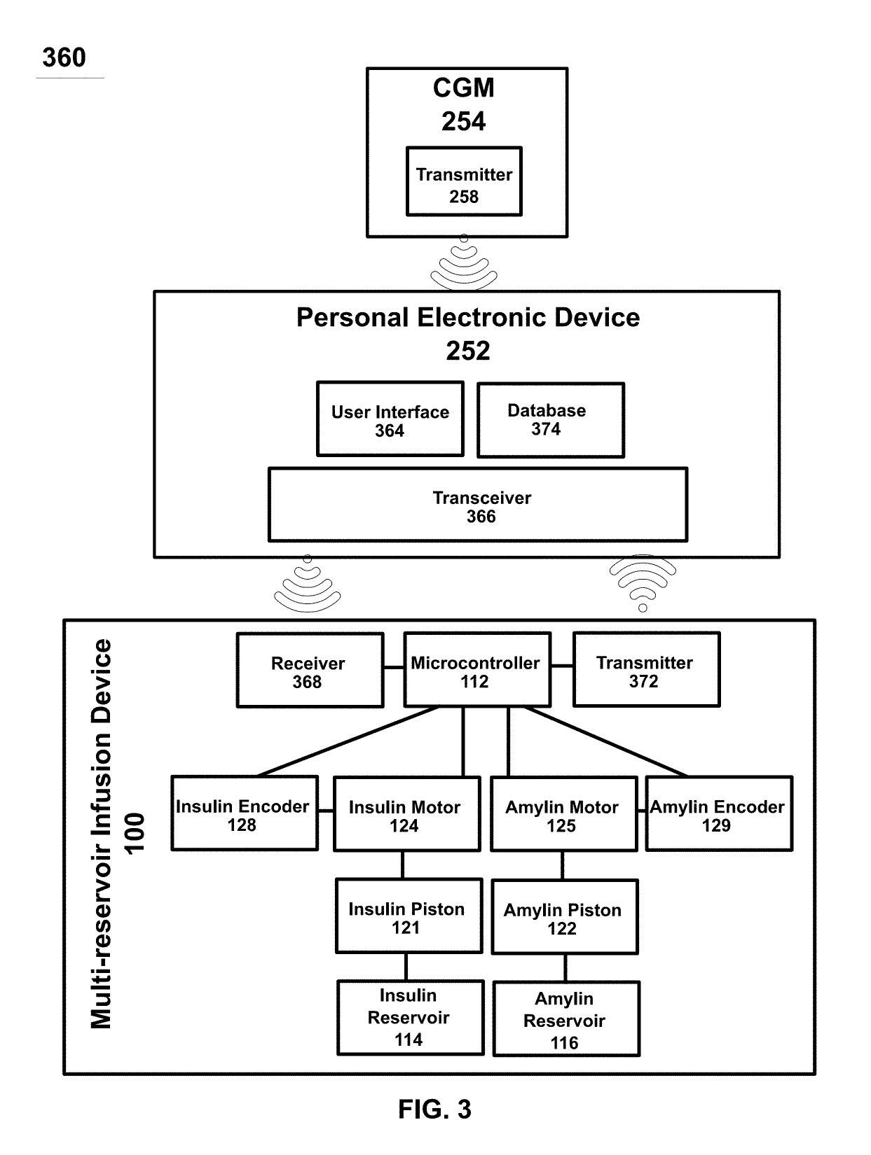

FIG. 3 is a functional block diagram for a medical infusion pump system 360, according to one embodiment. The medical infusion pump system 360 includes a CGM 254 that reads blood glucose levels. The CGM 254 may then wirelessly transmit the results of the blood glucose reading to a personal electronic device 252 via a wireless transmitter 258. The CGM 254 may transmit the blood glucose readings to the personal electronic device 252 continuously or periodically, according to various embodiments. Upon receiving the blood glucose reading, the personal electronic device 252 may indicate the results of the blood glucose reading via a user interface 364. If the blood glucose levels are above a target level, a processor module of the personal electronic device 252 may calculate the amount of active insulin in the bloodstream. In another embodiment, a user may calculate the levels of active insulin in the bloodstream manually an input instructions via the user interface 364. The amount of active insulin is determined by previous deliveries of insulin into the bloodstream.

If there is an insufficient amount of active insulin in the bloodstream to lower blood glucose levels to the target level, then another calculation is performed, either manually or by a processor module of the personal electronic device 252 itself, to determine how much insulin is needed to lower the blood glucose to the target level. If the calculation is performed manually, then the amount of insulin needed may be input into the personal electronic device 252 via the user interface 364. The personal electronic device 252 may wirelessly transmit the resulting calculation of insulin needed to lower blood glucose levels via a transceiver 366. The multi-reservoir infusion device 100 may receive the transmission from the personal electronic device 252 via a receiver 368. The receiver 368 may send an electrical signal to activate the microcontroller 112. Once activated, the microcontroller 112 may rotate an insulin motor 124 that corresponds to the insulin piston 121. The insulin piston 121 may apply pressure to the insulin reservoir 114 such that the insulin inside of the insulin reservoir 114 is dispensed from the multi-reservoir infusion device 100. An insulin encoder 128 may monitor the amount of insulin being dispensed from the insulin reservoir 114. When a dose of insulin sufficient to lower blood glucose to target levels has been dispensed, or when there is an occlusion, a transmitter 372 may transmit a signal to the transceiver 366 of the personal electronic device 252 to provide data for how much insulin was injected by the multi-reservoir infusion device 100. The transceiver 366 may send the data to a database 374 to later be used in future calculations to determine the amount of active insulin in the bloodstream.

Whenever levels of active insulin are calculated based on the levels of blood glucose, a processor module of the personal electronic device 252, or a user, may calculate what the blood glucose levels should have been based on previous injections of insulin. If the actual blood glucose levels are higher than expected levels, then the personal electronic device 252, or the user, may determine how much time has passed since the last injection of an amylin. If a sufficient amount of time has passed since the last injection of the amylin based on a predetermined timeframe, the personal electronic device 252 may send a signal via the transceiver 366 to the receiver 368 of the multi-reservoir infusion device 100. The receiver 368 may activate the microcontroller 112, which may rotate an amylin motor 125 corresponding to an amylin piston 122. The amylin piston 122 may apply pressure to the amylin reservoir 116 such that the amylin inside of the amylin reservoir 116 is dispensed from the multi-reservoir infusion device 100. An amylin encoder 129 may monitor the amount of insulin being dispensed from the amylin reservoir 116. The amount of amylin dispensed from the amylin reservoir 116 is an amount previously defined by the user and stored in a database 374 of the personal electronic device 252, or input by the user via the user interface 364. When the specified amount of amylin has been dispensed, or when there is an occlusion, the transmitter 372 may transmit a signal to the transceiver 366 of the personal electronic device 252 to provide data for how much amylin was injected by the multi-reservoir infusion device 100. The transceiver 372 may send the data to the database 374 to later be used in assessing the amount of time has passed since the last injection of amylin.

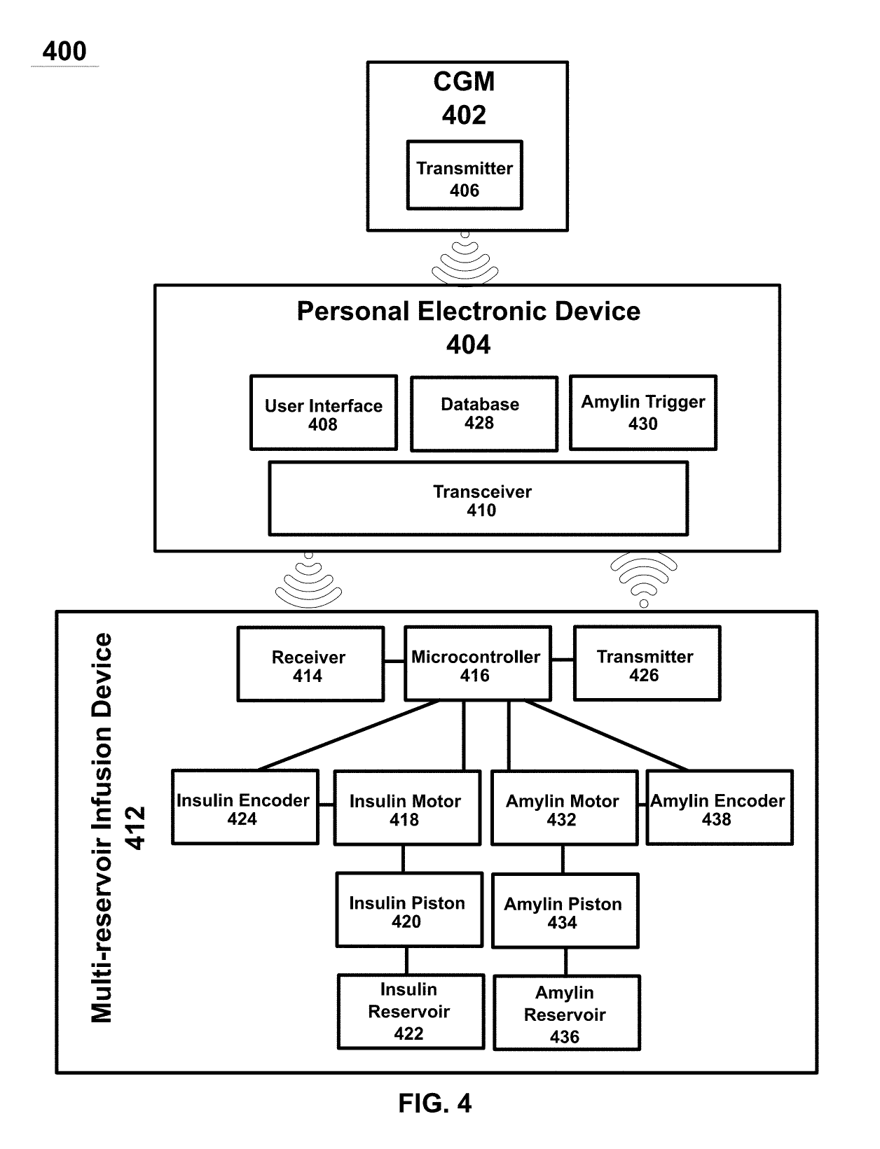

FIG. 4 is a functional block diagram for a medical infusion pump system 400, according to another embodiment. The medical infusion pump system 400 includes a CGM 402 that reads blood glucose levels. The CGM 402 may then wirelessly transmit the results of the blood glucose reading to a personal electronic device 404 via a wireless transmitter 406. The CGM 402 may transmit the blood glucose readings to the personal electronic device 404 continuously or periodically, according to various embodiments. Upon receiving the blood glucose reading, the personal electronic device 404 may indicate the results of the blood glucose reading via a user interface 408. If the blood glucose levels are above a target level, the personal electronic device 404 may calculate the amount of active insulin in the bloodstream. In another embodiment, a user may calculate the levels of active insulin in the bloodstream manually. The amount of active insulin is determined by previous deliveries of insulin into the bloodstream.

If there is an insufficient amount of active insulin in the bloodstream to lower blood glucose levels to the target level, then another calculation is performed, either manually or by a processor module on the personal electronic device 404 itself, to determine how much insulin is needed to lower the blood glucose to the target level. If the calculation is performed manually, then the amount of insulin needed may be input into the personal electronic device 404 via the user interface 408. The personal electronic device 404 may wirelessly transmit the resulting calculation of insulin needed to lower blood glucose levels via a transceiver 410. A multi-reservoir infusion device 412 may receive the transmission from the personal electronic device 404 via a receiver 414. The receiver 414 may send an electrical signal to activate the microcontroller 416. Once activated, the microcontroller 416 may rotate an insulin motor 418 that corresponds to the insulin piston 420. The insulin piston 420 may apply pressure to the insulin reservoir 422 such that the insulin inside of the insulin reservoir 422 is dispensed from the multi-reservoir infusion device 412. An insulin encoder 424 may monitor the amount of insulin being dispensed from the insulin reservoir 422. When a dose of insulin sufficient to lower blood glucose to target levels has been dispensed, or when there is an occlusion, a transmitter 426 may transmit a signal to the transceiver 410 of the personal electronic device 404 to provide data for how much insulin was injected by the multi-reservoir infusion device 412. The transceiver 410 may send the data to a database 428 to later be used in future calculations to determine the amount of active insulin in the bloodstream.

The personal electronic device 404 may include an amylin trigger 430. The amylin trigger 430 may provide an output for automatically dispensing a dose of amylin to a subject based on predicted and/or actual levels of blood glucose at a given time. The amylin trigger 430 may be in communication with the database 428 where data about previous blood glucose trends is stored and from which predictions of future blood-glucose levels are derived. The dose of amylin injected is independent of an amount of insulin dispensed into the subject. The amylin trigger's 430 output may prompt the personal electronic device 404 to send a signal via the transceiver 410 to the receiver 414 of the multi-reservoir infusion device 412. The receiver 414 may activate the microcontroller 416, which may rotate an amylin motor 432 corresponding to an amylin piston 434. The amylin piston 434 may apply pressure to the amylin reservoir 436 such that the amylin inside of the amylin reservoir 436 is dispensed from the multi-reservoir infusion device 412. An amylin encoder 438 may monitor the amount of amylin being dispensed from the amylin reservoir 436. The amount of amylin dispensed from the amylin reservoir 436 is an amount previously defined by the user and stored in the database 428 of the personal electronic device 404 or input by the user via the user interface 408.

In some cases, well-known features, structures or operations are not shown or described in detail. Furthermore, the described features, structures, or operations may be combined in any suitable manner in one or more embodiments. The embodiments of disclosed herein may be best understood by reference to the drawings, wherein like parts are designated by like numerals throughout. It will be readily understood that the components of the disclosed embodiments, as generally described and illustrated in the figures herein, could be arranged and designed in a wide variety of different configurations. Thus, the embodiments of the disclosure are not intended to limit the scope of the disclosure, as claimed, but are merely representative of possible embodiments of the disclosure.

Embodiments may be provided as a computer program product including a non-transitory computer and/or machine-readable medium having stored thereon instructions that may be used to program a computer (or other electronic device) to perform processes described herein. The non-transitory computer-readable medium may include, but is not limited to, hard drives, floppy diskettes, optical disks, CD-ROMs, DVD-ROMs, ROMs, RAMs, EPROMs, EEPROMs, magnetic or optical cards, solid-state memory devices, or other types of media/machine-readable medium suitable for storing electronic and/or processor executable instructions.

It should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims are hereby expressly incorporated into this Detailed Description, with each claim standing on its own as a separate embodiment. This disclosure includes all permutations of the independent claims with their dependent claims.

It will be apparent to those having skill in the art that changes may be made to the details of the above-described embodiments without departing from the underlying principles of the invention. Embodiments of the invention in which an exclusive property or privilege is claimed are defined as follows.

What is claimed is:

1. An medical infusion pump system comprising:
a multi-reservoir infusion device for dispensing at least compositions of insulin and an amylin analog for treatment of subjects, wherein insulin is stored in at least one insulin reservoir and the amylin analog stored in at least one amylin analog reservoir;
at least two positive displacement pumps for providing discharge pressure to the one or more insulin reservoirs and the one or more amylin analog reservoirs, wherein the two or more positive displacement pumps act independently;
an internal power source;
one or more motors; and
a noninvasive blood-glucose monitoring mechanism for continually monitoring glucose levels in a subject's blood, wherein the amylin analog is automatically dispensed into the subject's blood once the subject's blood-glucose levels rise above an expected threshold determined by previous and current glucose readings and/or an amount of insulin previously delivered to the subject.

2. The infusion pump system of claim 1 further comprising a processor module for calculating how much insulin to automatically inject based on expected carbohydrate intake, blood sugar level, still-active insulin and/or other indicators that determine how much insulin the subject needs.

3. The infusion pump system of claim 2 further comprising a database for recording a record of insulin and amylin analog usage, wherein the record is utilized by the processor module.

4. The infusion pump system of claim 2 further comprising a computation module for computing future blood-glucose levels.

5. The infusion pump system of claim 2 further comprising an amylin analog monitor for determining how much of the amylin analog has been injected and adjusting future insulin dose levels based on an amount of injected amylin analog.

6. The infusion pump system of claim 1 further comprising a timer for monitoring an amount of time elapsed after insulin and/or amylin analog delivery.

7. The infusion pump system of claim 1 further comprising a carbohydrate counter.

8. The infusion pump system of claim 1 further comprising an infusion set comprising a cannula for subcutaneous insertion and a tubing system for interfacing the insulin reservoir and/or amylin analog reservoir to the cannula.

9. The infusion pump system of claim 8, wherein the infusion set, insulin reservoir, and/or amylin analog reservoir are disposable.

10. The infusion pump system of claim 1 further comprising a programmed basal rate profile for insulin delivery.

11. The infusion pump system of claim 1 further comprising an adjuster, wherein the adjuster automatically injects a bolus of insulin and/or amylin in response to real time continuous glucose data received from the blood-glucose monitoring mechanism.

12. The infusion pump system of claim 1 further comprising an alert mechanism for alerting a subject of any malfunctions and/or other irregularities in the pump system's performance.

13. The infusion pump system of claim 1 further comprising a wireless communication system.

14. The infusion pump system of claim 13, wherein the wireless communication system comprises a personal electronic device with a user interface.

15. The infusion pump system of claim 14 further comprising a software application for receiving user inputs and transmitting user outputs.

16. The infusion pump system of claim 1, wherein the expected threshold is manually set by the subject and/or a medical professional.

17. The infusion pump system of claim 1 further comprising an automated tracker for tracking a subject's previous blood-glucose patterns and preprogramming future insulin and/or amylin analog quantities based on the patterns.

18. An medical infusion pump system comprising:
a multi-reservoir infusion device for dispensing at least compositions of insulin and an amylin analog for treatment of subjects, wherein insulin is stored in an insulin reservoir and the amylin analog stored in an amylin analog reservoir;
at least two positive displacement pumps for providing discharge pressure to the insulin reservoir and the amylin analog reservoir, wherein the two or more positive displacement pumps act independently;
an internal power source;
one or more motors; and
a noninvasive blood-glucose monitoring mechanism for continually monitoring glucose levels in a subject's blood, and
an adjuster module for automatically dispensing a dose of amylin analog, wherein the quantity of amylin analog dispensed is based on predicted levels of blood glucose and independent of an amount of insulin dispensed into the subject.

19. The infusion pump system of claim 18, wherein the adjuster module is in communication with a memory storage for storing data about previous blood glucose trends and from which predictions of future blood-glucose levels are derived.

20. The infusion pump system of claim 18 further comprising a timer for monitoring an amount of time elapsed after insulin and/or amylin analog delivery.
